(19) United States
(12) Patent Application Publication
(10) Pub. No.: US 2017/0124256 A1
(43) Pub. Date: May 4, 2017

(54) METHOD AND SYSTEM FOR ANALYZING ELECTROCARDIOGRAPH DATA

(71) Applicant: General Electric Company, Schenectady, NY (US)

(72) Inventors: Brian W. Nantz, Milwaukee, WI (US); Brian Joseph Young, Wauwatosa, WI (US)

(73) Assignee: General Electric Company, Schenectady, NY (US)

(21) Appl. No.: 14/927,593

(22) Filed: Oct. 30, 2015

(51) Int. Cl. G06F 19/00; A61B 5/00; A61B 5/0452

(52) U.S. Cl. CPC G06F 19/322; A61B 5/0452; A61B 5/7221; G06F 19/366

(57) ABSTRACT

A method for analyzing ECG data includes recording a biometric identifier from a patient and encoding the biometric identifier to create a patient's encoded biometric identifier. The method further includes accessing an ECG database of prior ECG files, each prior ECG file containing a prior ECG waveform and a prior encoded biometric identifier therein. The method further includes comparing the patient's encoded biometric identifier to the prior encoded biometric identifier of one or more of the prior ECG files to locate a matching prior ECG file containing the prior encoded biometric identifier that matches the patient's encoded biometric identifier, and then presenting the matching prior ECG file.

### BACKGROUND

The present invention relates to a method and system for analyzing electrocardiograph (ECG) data utilizing biometrics to identify prior ECG recordings for a patient.

Electrocardiograph (ECG) monitoring is a routine part of patient care, including emergency care. Often, an important part of providing a clinical assessment of an ECG waveform includes comparison of ECG waveforms taken over time, such as comparing a current waveform for a patient to a waveform for that patient taken on a prior date. Significant information may often be gleaned by comparing a patient's current ECG waveforms to one or more previously-recorded waveforms for that patient.

ECG recordings taken for a patient are routinely stored as part of a patient's medical record, and are also routinely stored in ECG databases, such as the MUSE ECG management system produced by General Electric Company of Schenectady, N.Y. Such systems require identification of a patient, such as by patient's name, social security number, and/or other identification number, in order to access a patient's prior ECG waveforms. Access to a patient's medical record may not be possible in an emergency situation, where a patient may be physically and/or mentally incapacitated, or otherwise unable to identify themselves. In such situations, clinicians are required to interpret a patient's current ECG waveforms without the benefit of having a prior ECG waveform with which to compare the current ECG. Accordingly, important information may be missed and/or a patient may be misdiagnosed due to the lack of information available to the clinician at the time of reading the ECG.

Presently, technologies are available that utilize biometrics to identify patients in medical applications to prevent patient misidentification and/or fraud. Such systems generally contain biometric databases that link biometric identifiers to other patient identification information, such as name, social security number, medical record number, etc. Thus, in prior art systems, biometrics are used to identify patients by comparing the results of a biometric scan to a biometric database. Then, once a patient has been identified by name, social security, etc., that patient identification information is used to access the patient's medical records. However, these existing systems using biometrics to get patient identification information are bulky and require significant infrastructure and security, all of which limits their broad usage and adoption. Moreover, due at least in part to security concerns, as well as HIPPAA and JCAHO regulations, access to such systems is severely limited because of security and patient confidentiality concerns. Accordingly, access to databases of patient biometric identification data and patient medical record data must be tightly controlled and not widely granted.

### SUMMARY

The present disclosure includes a system and method for analyzing electrocardiograph (ECG) data that overcomes the problems and challenges described above. This summary is provided to introduce a selection of concepts that are further described below in the Detailed Description. This Summary is not intended to identify key or central features of the claimed subject matter, nor is it intended to be used as an aid in limiting the scope of the claimed subject matter.

A method for analyzing ECG data includes recording a biometric identifier from a patient and encoding the biometric identifier to create a patient's encoded biometric identifier. The method further includes accessing an ECG database of prior ECG files, each prior ECG file containing a prior ECG waveform and a prior encoded biometric identifier therein. The method further includes comparing the patient's encoded biometric identifier to the prior encoded biometric identifier of one or more of the prior ECG files to locate a matching prior ECG file containing the prior encoded biometric identifier that matches the patient's encoded biometric identifier, and then presenting the matching prior ECG file.

One embodiment of a system for analyzing ECG data includes an ECG database containing prior ECG files, each ECG file having a prior ECG waveform and a prior encoded biometric identifier therein. The system further includes a biometric scanner that senses a patient's biometric identifier, and an encoder module that receives and encodes the patient's biometric identifier to create a patient's encoded biometric identifier. The system further includes a processor and an ECG matching module that is executable on the processor to access an ECG database containing prior ECG files, each ECG file containing a prior ECG waveform and a prior encoded biometric identifier therein. The ECG matching module further compares the patient's biometric identifier to the prior encoded biometric identifier to locate a matching prior ECG file containing the prior encoded biometric identifier that matches the patient's encoded biometric identifier. The ECG matching module then presents the matching prior ECG file.

In one embodiment, a non-transitory computer readable medium having computer-executable instructions stored thereon has instruction including the steps of recording a biometric identifier from a patient and encoding the biometric identifier to create a patient's encoded biometric identifier. The steps further include recording a new ECG waveform from the patient and creating a new ECG file containing the new ECG waveform and the patient's coded biometric identifier. The steps further include accessing an ECG database of prior ECG files, each ECG file containing a prior ECG waveform and a prior encoded biometric identifier therein. The steps further include comparing the patient's encoded biometric identifier to the prior encoded biometric identifier to locate a prior ECG file containing the prior encoded biometric identifier that matches the patient's encoded biometric identifier, and then presenting the matching prior ECG file.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 schematically depicts one embodiment of a system for analyzing ECG data.

FIG. 2 schematically depicts another embodiment of a system for analyzing ECG data.

FIG. 3 depicts one embodiment of an ECG file for a patient according to the present disclosure.

FIG. 4 depicts another embodiment of an ECG file for the patient according to the present disclosure.

FIG. 5 is a flowchart depicting one embodiment of a method for analyzing ECG data according to the present disclosure.

FIG. 6 depicts another embodiment of a method for analyzing ECG data according to the present disclosure.

### DETAILED DESCRIPTION

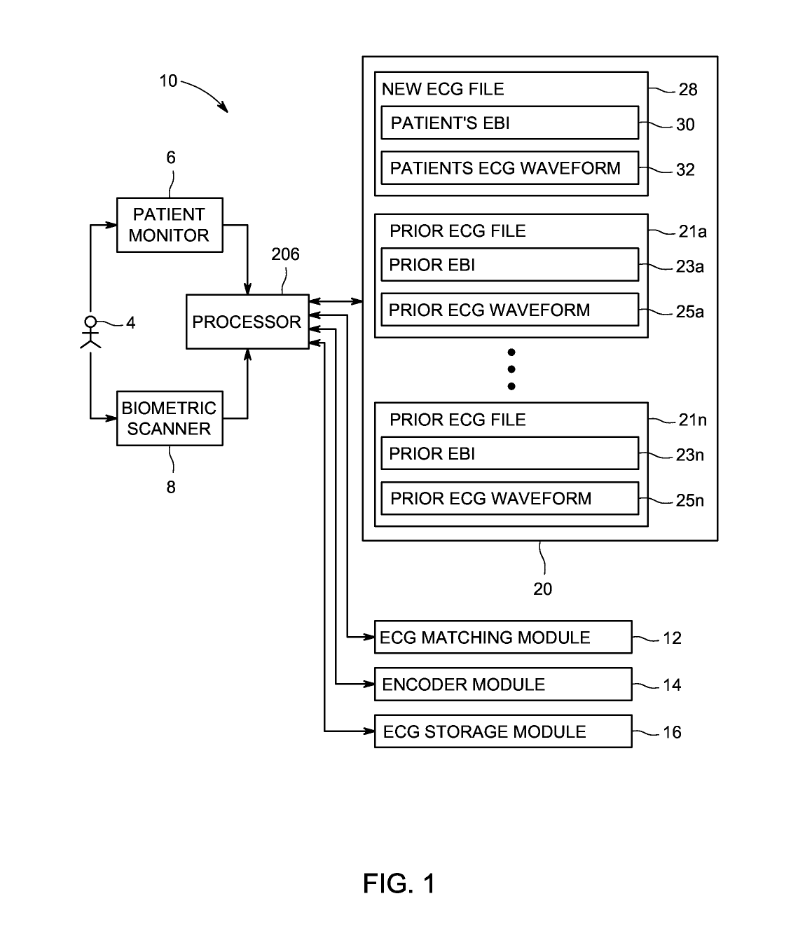

FIG. 1 depicts one embodiment of a system 10 for analyzing ECG data. The system 10 and method 50 for analyzing ECG data disclosed herein overcomes the problems and challenges discussed above relating to accessing prior ECG waveforms for a patient. The present inventors have developed the disclosed system 10 and method 50 disclosed herein to overcome the above-mentioned problems with prior art systems involving biometric identification. Through their research and development in the field, the present inventors have recognized the need for a system and method for analyzing electrocardiograph (ECG) data that allows identification and access to prior ECG recordings for a patient without requiring patient identification, such as patient name or patient identification number. Furthermore, the inventors have recognized that a system for prior ECG identification is desirable that may be broadly implemented without encountering at least some of the confidentiality and security concerns associated with individually identifiable health information, and that allows quick and immediate access to prior ECG data for the patient. Moreover, the present inventors have recognized that such a system for analysis of ECG data does not require access to a patient's full medical record, and that a self-contained system allowing identification of ECG data for a patient based only on the ECG files in an ECG database is desirable. For example, such a system may disassociate the biometric identifier from other identifying information for a patient, which may decrease security and patient confidentiality concerns. Moreover, such a self-contained system may be quicker and more efficient to use in emergency situations where identifying prior ECG data for a patient as quickly as possible is a priority.

A system 10 for analyzing ECG data may include an ECG database 20 of prior ECG files 21a-21n searchable based on an encoded biometric identifier (EBI) stored therein. An EBI 23a-23n for a patient is stored with an ECG waveform 25a-25n for that patient in a single ECG file 21a-21n. The EBI may be based on any anatomic features unique, or nearly unique, to each patient. These biometric identifiers include, for example, fingerprints, palm prints, palm veins, hand geometry, facial recognition, DNA, iris patterns, or retinal blood vessel patterns. Devices and technologies are available for scanning these portions of a patient's body and digitizing the scan, or some portion of the scan, to create an image or a data file depicting or describing the unique physiological makeup of the relevant portion of the patient's body.

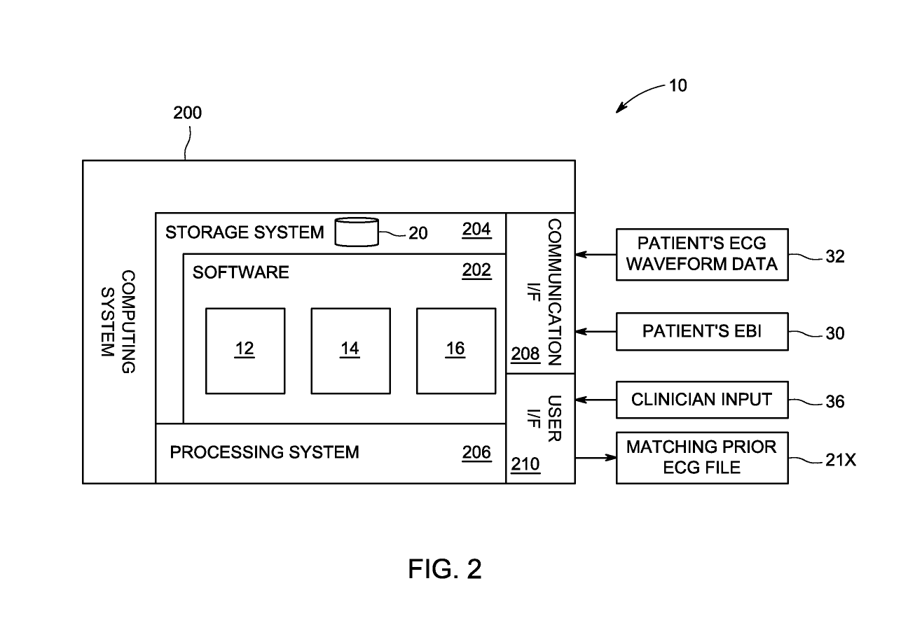

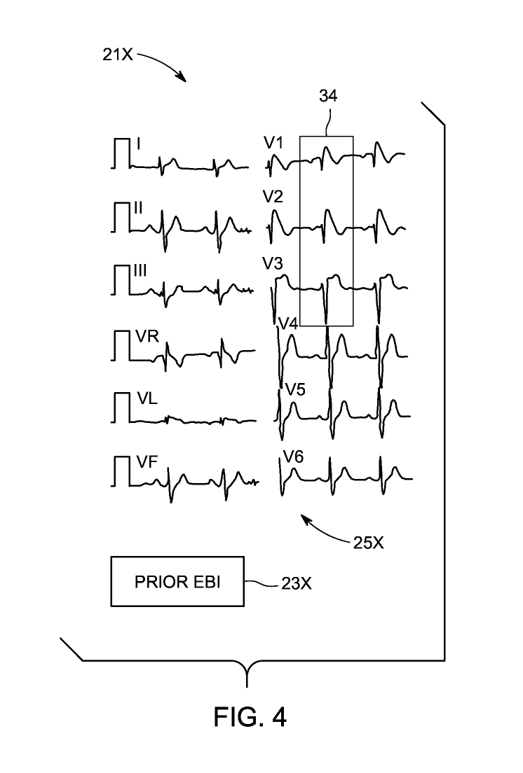

Accordingly, a biometric identifier may be recorded from a patient 4, such as with a biometric scanner 8, which may be any device or technology capable of capturing a biometric identifier from a patient. The biometric identifier may then be encoded in the same way that the prior EBIs 23a-23n are encoded in order to create an EBI 30 for the patient 4. The ECG database may then be searched based on the patient's EBI 30 in order to identify a prior ECG file for that patient 4. Specifically, an ECG matching module 12 may search the prior EBIs 23a-23n of each prior ECG file 21a-21n in the ECG database 20 in order to find a match for the patient's EBI 30. Numerous methods and systems are available in the art for biometric identification matching, and a person having ordinary skill in the relevant art will understand that any number of such biometric matching methods and systems may be utilized in the disclosed system. Furthermore, the ECG matching module may be configured to require an identical match between the patient's EBI 30 and a matching prior EBI 23a-23n. Alternatively, the ECG matching module 12 may be configured to identify a match between a prior EBI 23a-23n and the patient's EBI 30 when they share at least a predefined amount of features, such as a predefined number of features or a predefined percent of the total features. For example, the ECG matching module 12 may be configured to use fuzzy logic in order to identify matches, and the one or more identified matching prior ECG files 21x with matching prior EBIs 23x, which include prior ECG waveforms 25x, may be presented to a clinician for further analysis. (See FIGS. 2 and 4) In another embodiment, the ECG matching module 12 may require a certain amount of alignment or match between the prior EBI 23a-23n and the patient's EBI 30 in order to identify a match. Thus, in one embodiment, the goal of the system 10 is to identify all prior ECG files 21a-21n that could be prior recordings from the patient 4, and thus the system may err on the side of overinclusion and over identification of matches, rather than erring on the side of requiring that the prior EBI 23a-23n match identically with the patient's EBI 30 and thus run the risk of erroneously missing or eliminating a prior ECG file 21a-21n validly belonging to, or recorded from, the patient 4. In yet another embodiment, the ECG matching module 12 may require exact alignment or match between the prior EBI 23a-23n and the patient's EBI 30 in order to identify a match.

The EBI for a patient is stored in each ECG file 21, 28 for that patient, and it may be encoded into each prior ECG file 21a-21n in any number of ways. A person having ordinary skill in the relevant art will understand that various encoding methods may be appropriate. For example, the prior EBI 23a-23n may be embedded in each prior ECG file 21a-21n as metadata or as a data element tag. To provide one exemplary embodiment, each prior ECG file 21a-21n may be a 12-lead diagnostic DICOM file, and the prior EBI 23a-23n may be stored in the DICOM file as a private data element tag. Alternatively, another embodiment is use of a binary format that is proprietary to a manufacturer, which may contain the EBI as a specific data element.

Raw biometric data from a biometric scanner 8, such as image data, may be stored in the prior ECG file 21a-21n. However, there are multiple drawbacks to storing the raw biometric data in the prior ECG file 21a-21n. Firstly, raw biometric data, such as an image of the relevant aspect of the patient's body, may create more security, privacy, and patient confidentiality concerns than an encoded version of the information. The encoded data may present fewer security, confidentiality, and privacy concerns, as it may capture only a select portion of the patient's biometric identifier and may do so in a way that is not easily interpreted by outside sources. Moreover, the encoded biometric identifiers may further be encrypted to provide further security, and thus the encoding process may include encryption. Further, raw biometric data takes up a significant amount of space, and encoding the biometric data significantly reduces the amount of space required to store the biometric identifier. For example, a raw fingerprint image may take up about 250 KB of space, whereas an encoded version may only require 500 bytes of space or less. Furthermore, analysis and comparison of biometric identifiers in their raw form, such as to compare the patient's EBI 30 to prior EBIs 23a-23n, would require significantly more computing power and would take more time to execute than the same comparison of encoded data. Thus, the biometric identifiers may be encoded to quickly allow comparison with the patient's EBI 30.

Multiple encoding methods and algorithms for biometric identifier data are known in the relevant art, and a person having ordinary skill in the relevant art will understand in light of this disclosure that the encoding may be performed by any number of known methods. Standards exist for encoding biometrics, such as those developed by the International Organization for Standardization (ISO). For example, in an embodiment utilizing fingerprints or palm prints as biometric identifiers, the EBIs 23, 28 may be encoded according to ISO 19794-2 or ISO 19794-4 specifications. Alternatively, the EBIs 23, 28 may be created using a non-standard, proprietary, or secret encoding algorithm and method, which may allow complete disassociation between the EBI 30 for the patient and other identifying information for that patient.

The encoder module 14 is configured to encode the biometric identifier in order to create the EBI stored in each associated ECG file. In one embodiment, the encoder module 14 may detect and isolate a set of features within the biometric identifier and encode each of the features to create the EBI. For example, the encoder module 14 may be configured to isolate and encode a predefined portion of a patient's biometric identifier. For example, in an embodiment using a fingerprint biometric identifier, the encoder module 14 may be configured to isolate and encode a predefined portion of the raw image data of the patient's fingerprint around the center of the arch, loop, or whirl pattern of the fingerprint. Alternatively or additionally, the encoder module 14 may be configured to detect a certain set of minutiae features within the biometric identifier data, and only encode those particular features. In context of the fingerprint example, the encoder module 14 may be configured to focus on one or more major minutiae features of fingerprint ridges, such as ridge endings, bifurcations, and short ridges, and the location thereof relative to some predefined, identifiable location within the fingerprint. By limiting the number of features encoded, the space required for storing the EBI may be reduced and the comparison process with the patient's EBI 30 may be expedited. Moreover, the predefined features may be ordered in a predefined way so that the EBIs may be easily comparable to one another.

The encoder module 14 and the ECG module 12, along with the ECG storage module 16, are executed by the processor 206 in order to affect the method 50 for analyzing ECG data described and disclosed herein. More specifically, the encoder module 14 receives a biometric identifier for the patient 4 from the biometric scanner 8. The biometric scanner 8 may be any device capable of scanning the patient's relevant anatomy in order to record the biometric identifier. In an embodiment where the biometric identifier is a fingerprint, palm print, or palm geometry, the biometric scanner 8 may be, for example, a scanner utilizing optical imaging, ultrasonic imaging, or capacitance sensors in order to form fingerprint or palm print images. In other embodiments, the biometric scanner 8 may be a retina scanner to scan the unique vessel pattern in the patient's retina, or an iris scanner to scan and image the iris of the patient's eye. The retinal or iris scanners may be, for example, infrared or near infrared imagers configured to be placed in close proximity of the patient's eye. Alternatively, in an embodiment where the biometric scanner 8 is an iris scanner, the iris scanner may utilize light in the visible spectrum in order to create the image of the patient's iris. Moreover, the biometric scanner 8 may be a stand-alone device, or it may be integrated into another portion of the system 10, such as the patient monitor 6 may be any ECG monitoring device capable of recording an ECG waveform from the patient.

Referring to the embodiment of FIG. 1, once the biometric scanner 8 has scanned the biometric identifier from the patient 4, the encoder module 14 is engaged on the processor 206 in order to encode the biometric identifier as described above. Accordingly, the patient's EBI 30 is created. Likewise, each prior EBI 23a-23n is created similarly. The patient's EBI 30 is then sent to the ECG matching module 12, which compares the patient's EBI 30 to the prior EBIs 23a-23n available in the ECG database 20 to identify matches. In an embodiment where the patient's EBI 30 is comprised of distinct features, the features from the patient's EBI 30 may be compared to corresponding features of the prior EBI 23a. The ECG matching module 12 may require that the patient's EBI 30 match identically to the prior EBI 23a-23n in order to declare a match. Alternatively, the ECG matching module 12 may identify a match where the patient's EBI 30 shares at least a predefined number or percentage of features with the prior EBI 23a-23n. For example, a match may be declared where 90% or higher of the encoded features in the patient's EBI 30 are identified in the prior EBI 23a. The predefined amount of shared features may be adjusted higher or lower depending on the need and operation of the system. Where certainty of match is required, the predefined amount of shared features may be set closer to 100%. In other embodiments where the goal of the system 10 is to identify any possible ECG waveform that could belong to the patient, the predefined amount of shared features may be set lower. Where matches are identified between the patient's EBI 30 and a prior EBI 23a-23n, the relevant prior EBI is deemed to be matching EBI 23x and the associated prior ECG file 21a-21n is deemed to be a matching prior ECG file 21x. (See FIGS. 2 and 4) The one or more matching prior ECG files 21x are then presented as outputs of the ECG matching module 12, which may then be displayed to a clinician on a user interface device 210.

In certain embodiments, and especially in embodiments where the predefined amount of shared features in order to identify a match is set low, the one or more identified matching prior ECG files 21x may be further assessed in order to determine whether the matching prior ECG file 21x contains a prior ECG waveform 25x for the patient 4. For example, the matching prior ECG waveforms 25x may be compared to the patient's ECG waveform 32, which has been recorded from the patient 4 at the patient monitor 6. The waveforms may be compared, for example, to see if any distinguishing features may be identified in the ECG waveforms 25, 32 that are common to both waveforms.

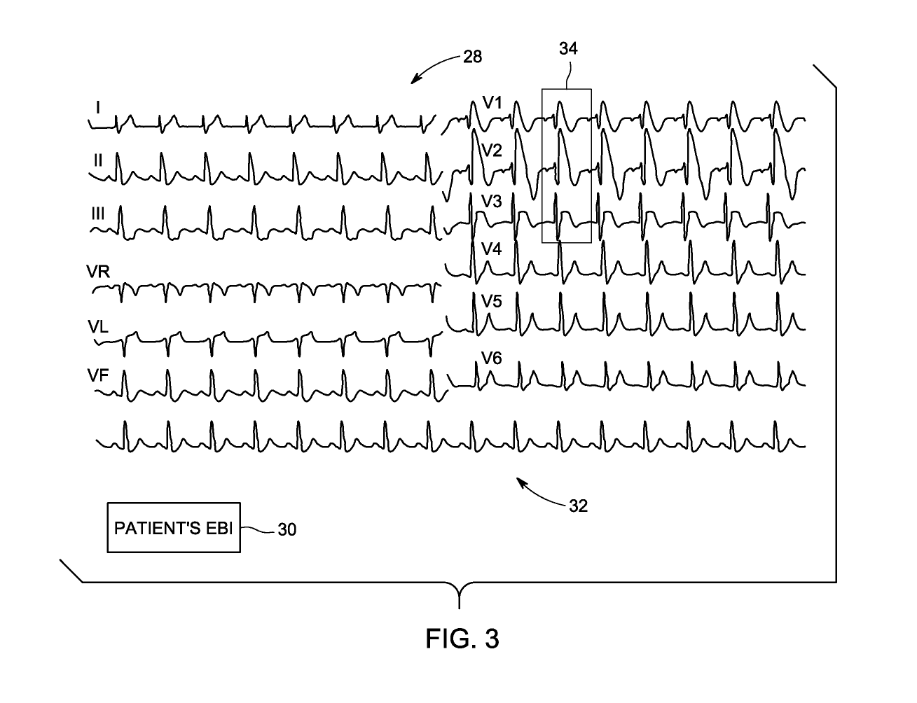

In one embodiment, the ECG matching module 12 may be configured to automatically compare the matching prior ECG waveform 25x to the patient's ECG waveform 32 in order to confirm that the matching prior ECG file 21x contains ECG waveforms of the patient 4. For example, as is illustrated with respect to FIGS. 3 and 4, the ECG matching module 12 may be configured to detect distinguishing features 34 that are common to both the patient's ECG waveform 32 and the matching prior ECG waveform 25x. FIG. 3 illustrates an exemplary new ECG file 28 created for the patient containing the patient's EBI 30 and the patient's ECG waveform 32. FIG. 4 illustrates a matching prior ECG file 21x containing a prior EBI 23x and a prior ECG waveform 25x. A distinguishing feature 34 exists in both the patient's ECG waveform 32 and the prior ECG waveform 25x. In the exemplary embodiment, the distinguishing feature 34 includes an ST elevation in leads V1, V2, and V3. This distinguishing feature 34 appears similarly in both the patient's ECG waveform 32 and the prior ECG waveform 25x identified as a match, and the presence of the common distinguishing feature 34 adds to the certainty that the identified matching prior ECG file 21x actually does contain a prior ECG waveform for the patient 4. Any number of morphological features and/or rhythm features that are sufficiently uncommon to provide at least some verifying information that the matching prior ECG waveform 25X actually belongs to the patient 4 may be used as distinguishing features 4. Other examples of patterns that may be considered a distinguishing feature in an individual's ECG are patterns including early repolarization, delta waves due to Wolff-Parkinson-White syndrome and a short PR interval due to Lown-Ganong-Levine syndrome.

Alternatively or additionally, the one or more matching prior ECG files 21x, and specifically the prior ECG waveforms 25x therein, may be presented to a clinician, and the clinician may conduct the comparison between waveforms to identify distinguishing features 34 there between. The clinician may further provide input 36 to the system 10 that may be utilized by the ECG matching module 12 confirm or deny each identified matching prior ECG file 21x. Thus, the ECG matching module 12 may act in conjunction with the clinician input 36 to identify distinguishing features 34 in the waveforms and confirm the match. For example, the clinician input 36 may identify certain matching ECG files 21x, aspects of the matching prior ECG waveform 25x, and/or aspects of the patient's ECG waveform 30 to be compared by the ECG matching module 12 to confirm the match.

Whether or not a match is located, the ECG storage module 16 may create a new ECG file 28 containing the patient's EBI 30 and the patient's ECG waveform 32, such as the waveform recorded at patient monitor 6. The new ECG file may be added to the ECG database 20 so that it may be searched at a later date, and may eventually provide a prior ECG waveform for the patient 4. In this way, the ECG database 20 can be built and maintained.

In one embodiment, a feature of this system 10 and method 50 utilizing the EBIs may provide an ability to associate ECG waveforms for a patient together without actually identifying the patient, such as knowing any information normally used in medical settings to identify a patient—e.g., name, personal identification number(s), demographic information, address(es), etc. As described above, the method of identifying matching prior ECG files 21x does not require obtaining any identifying information from the patient other than the biometric scan, and thus the identification of prior ECG waveforms may happen regardless of the patient's ability to identify themselves due to impaired mental or physical state. Furthermore, the system 10 may be configured and operated so that no direct link is ever made between the patient's EBI 30 and other identifying information for the patient, such as a patient's name, social security number, medical record number, etc. In other words, in one possible embodiment, the system 10 does not draw any link between the EBI 23, 30 and patient identification information. Moreover, as discussed above, the patient's EBI 30 may contain only a portion of the patient's biometric identifier, which may go even further to ease security concerns regarding safeguarding patient privacy and identification information. Accordingly, the prior ECG waveforms for a patient may be indexed and identifiable without requiring storage and access to confidential information that could be used to identify the patient.

Alternatively, in another embodiment of the system, the system may be configured to present the prior ECG waveform along with information about the patient—such as age, gender, and race—that the responsible party for the system may be able to present to a clinical user without compromising confidentiality requirements. With this information, the user may be able to confirm the ECG is from the patient based on the consistency of the additional patient information with the patient presentation.

FIG. 2 provides another system diagram of an exemplary embodiment of the system 10 for analyzing ECG data having an ECG database 20 storing prior ECG waveform data 25 and prior EBIs 23, and having an ECG matching module 12 executable to provide the comparisons and identify matching ECG files 21x, as well as having an encoder module 14 and ECG storage module 16 that operate as described herein. The system 10 is generally a computing system 200 that includes a processing system 206, storage system 204, software 202, communication interface 208 and a user interface 210. The processing system 206 loads and executes software 202 from the storage system 204, including the ECG matching module 12, encoder module 14, and ECG storage module 16, which are applications within the software 202. Each of the modules 12, 14, 16 include computer-readable instructions that, when executed by the computing system 10 (including the processing system 206), direct the processing system 206 to operate as described in herein in further detail, including to execute the steps to locate a matching prior ECG file 21x containing a prior EBI 23x that matches the patient's EBI 30.

Although the computing system 10 as depicted in FIG. 2 includes one software 202 encapsulating one comparator module 24, one encoder module 14, and one ECG storage module 16, it should be understood that one or more software elements having one or more modules may provide the same operation. Similarly, while description as provided herein refers to a computing system 200 and a processing system 206, it is to be recognized that implementations of such systems can be performed using one or more processors, which may be communicatively connected, and such implementations are considered to be within the scope of the description.

The processing system 206 can comprise a microprocessor and other circuitry that retrieves and executes software 202 from storage system 204. Processing system 206 can be implemented within a single processing device but can also be distributed across multiple processing devices or sub-systems that cooperate in existing program instructions. Examples of processing system 206 include general purpose central processing units, applications specific processors, and logic devices, as well as any other type of processing device, combinations of processing devices, or variations thereof.

The storage system 204, which includes the ECG database 20, can comprise any storage media, or group of storage media, readable by processing system 206, and capable of storing software 202. The storage system 204 can include volatile and non-volatile, removable and non-removable media implemented in any method or technology for storage of information, such as computer-readable instructions, data structures, program modules, or other data. Storage system 204 can be implemented as a single storage device but may also be implemented across multiple storage devices or sub-systems. For example, the software 202 may be stored on a separate storage device than the ECG database 20. Likewise, ECG database 20 can be stored, distributed, and/or implemented across one or more storage media or group of storage medias. Similarly, ECG database 20 may encompass multiple different sub-databases at different storage locations and/or containing different information which may be stored in different formats. By way of example, the ECG database 20 may encompass a MUSE ECG management system housing waveform data. Storage system 204 can further include additional elements, such a controller capable, of communicating with the processing system 206.

Examples of storage media include random access memory, read only memory, magnetic discs, optical discs, flash memory, virtual memory, and non-virtual memory, magnetic sets, magnetic tape, magnetic disc storage or other magnetic storage devices, or any other medium which can be used to storage the desired information and that may be accessed by an instruction execution system, as well as any combination or variation thereof, or any other type of storage medium. Likewise, the storage media may be housed locally with the processing system 206, or may be distributed in one or more servers, which may be at multiple locations and networked, such as in cloud computing applications and systems. In some implementations, the store media can be a non-transitory storage media. In some implementations, at least a portion of the storage media may be transitory.

The user interface 210 is configured to receive input 36 from a clinician, and to produce one or more matching prior ECG files 21x to the clinician. User interface 210 may include a mouse, a keyboard, a voice input device, a touch input device for receiving a gesture from a user, a motion input device for detecting non-touch gestures and other motions by a user, and other comparable input devices and associated processing elements capable of receiving user input from a user, such as a clinician. Output devices such as a video display or graphical display can display an interface further associated with embodiments of the system and method as disclosed herein. Speakers, printers, haptic devices and other types of output devices may also be included in the user interface 210.

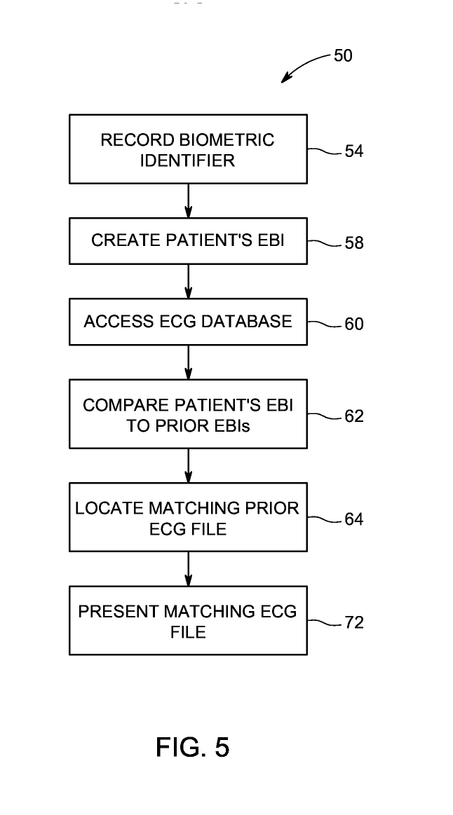

FIG. 5 depicts one embodiment of a method 50 for analyzing ECG data. A biometric identifier is recorded at step 54. A patient's EBI is then created at step 54 based on the recorded biometric identifier. At step 60, the ECG database is accessed, and then the patient's EBI is compared to the prior EBIs in the ECG database at step 62. A matching prior ECG file is located at step 64, and then the matching ECG file is presented at step 72.

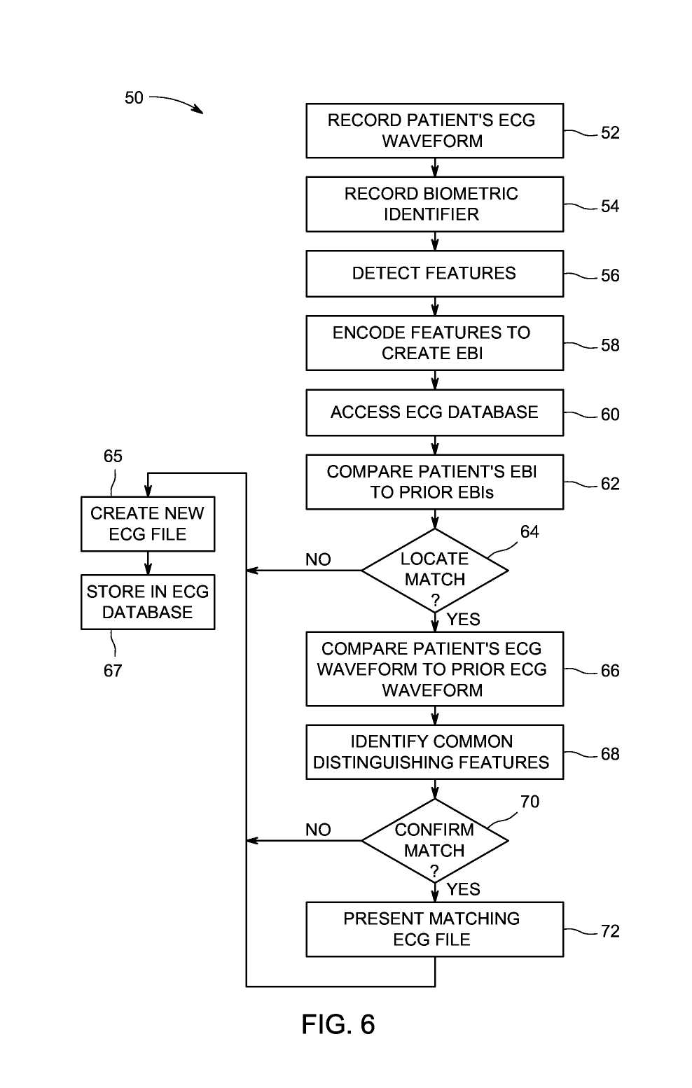

FIG. 6 depicts another embodiment of a method 50 for analyzing ECG data. At step 52, a patient's ECG waveform is recorded, such as via patient monitor 6. A biometric identifier is then recorded at step 54, such as via biometric scanner 8. At step 56, features are then detected in the biometric identifier data recorded at step 54. At step 58, the features are encoded in order to create a patient's EBI 30. The ECG database 20 is then accessed at step 60, and the patient's EBI 30 is then compared to the prior EBIs 23a-23n in the ECG database 20. If a match is located at step 64, then the patient's ECG waveform 32 is compared to the prior ECG waveform 25x in the identified matching ECG file identified at step 66. If a common distinguishing feature is identified at step 68, then a match is confirmed at step 70, and the matching ECG file 21x is presented at step 72.

A new ECG file 28 is created at step 65 which includes the patient's EBI 30 and the patient's ECG waveform 32. The new ECG file 28 is then stored in the ECG database 20 at step 67. Likewise, if a match is not located at step 64, or the match is not confirmed at step 70, the system 10 proceeds to step 65 to create the new ECG file 28 for the patient.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method for analyzing electrocardiograph (ECG) data, the method comprising:
recording a biometric identifier from a patient;
encoding the biometric identifier to create a patient's encoded biometric identifier;
accessing an ECG database of prior ECG files, each prior ECG file containing a prior ECG waveform and a prior encoded biometric identifier therein;
comparing the patient's encoded biometric identifier to the prior encoded biometric identifier of one or more of the prior ECG files to locate a matching prior ECG file containing the prior encoded biometric identifier that matches the patient's encoded biometric identifier; and
presenting the matching prior ECG file.

2. The method of claim 1, further comprising:
recording a patient's ECG waveform from the patient;
creating a new ECG file containing the patient's ECG waveform and the patient's encoded biometric identifier; and
storing the new ECG file in the ECG database.

3. The method of claim 1, wherein the matching prior ECG file contains a prior encoded biometric identifier that is identical to the patient's encoded biometric identifier.

4. The method of claim 1, wherein the step of encoding the patient's biometric identifier includes detecting a set of features of the biometric identifier and encoding each of the features to create the patient's encoded biometric identifier.

5. The method of claim 4, wherein the prior encoded biometric identifier of the matching prior ECG file shares at least a predefined amount of features with the patient's encoded biometric identifier.

6. The method of claim 2, further comprising comparing the prior ECG waveform to the patient's ECG waveform to confirm the matching prior ECG file.

7. The method of claim 1, wherein the biometric identifier is at least one of a fingerprint, a retina scan, and an iris scan.

8. A system for analyzing electrocardiograph (ECG) data, the system comprising:
an ECG database containing prior ECG files, each ECG file containing a prior ECG waveform and a prior encoded biometric identifier therein;
a biometric scanner that senses a patient's biometric identifier;
an encoder module that receives and encodes the patient's biometric identifier to create a patient's encoded biometric identifier;
a processor; and
an ECG matching module that, when executed on the processor:
accesses the ECG database;
compares the patient's encoded biometric identifier to the prior encoded biometric identifier to locate a matching prior ECG file containing the prior encoded biometric identifier that matches the patient's encoded biometric identifier; and
presents the matching prior ECG file.

9. The system of claim 8, further comprising:
an ECG monitor that records a patient's ECG waveform from the patient; and
an ECG storage module that creates a new ECG file containing the patient's ECG waveform and the patient's encoded biometric identifier, and stores the new ECG file in the ECG database.

10. The system of claim 8, wherein the matching prior ECG file contains a prior encoded biometric identifier that is identical to the patient's encoded biometric identifier.

11. The system of claim 8, wherein the encoder module detects a set of features of the biometric identifier and encodes each of the features to create the patient's encoded biometric identifier.

12. The system of claim 8, wherein the matching prior ECG file contains a prior encoded biometric identifier that shares at least a predefined amount of features with the patient's encoded biometric identifier.

13. The system of claim 8, wherein the ECG matching module further compares the prior ECG waveform to the patient's ECG waveform to confirm the matching prior ECG file.

14. The system of claim 8, wherein the biometric scanner is at least one of a fingerprint scanner, a palm scanner, a retina scanner, and an iris scanner.

15. A non-transitory computer readable medium having computer-executable instructions stored thereon, wherein the instructions include the steps comprising:
recording a biometric identifier from a patient;
encoding the biometric identifier to create a patient's encoded biometric identifier;
recording a patient's ECG waveform from the patient;
creating a new ECG file containing the patient's ECG waveform and the patient's encoded biometric identifier;
accessing an ECG database of prior ECG files, each ECG file containing a prior ECG waveform and a prior encoded biometric identifier therein;
comparing the patient's encoded biometric identifier to the prior encoded biometric identifier to locate a matching prior ECG file containing the prior encoded biometric identifier that matches the patient's encoded biometric identifier; and
presenting the matching prior ECG file.

16. The non-transitory computer readable medium of claim 15, wherein the instructions include the steps further comprising storing the new ECG file in the ECG database.

17. The non-transitory computer readable medium of claim 15, wherein the matching prior ECG file contains a prior encoded biometric identifier that is identical to the patient's encoded biometric identifier.

18. The non-transitory computer readable medium of claim 17, wherein the step of encoding the patient's biometric identifier includes detecting a set of features of the biometric identifier and encoding each of the features to create the patient's encoded biometric identifier.

19. The non-transitory computer readable medium of claim 18, wherein the matching prior ECG file contains a prior encoded biometric identifier that shares at least a predefined number of features with the patient's encoded biometric identifier.

20. The non-transitory computer readable medium of claim 15, wherein the instructions include the steps further comprising comparing the prior ECG waveform to the patient's ECG waveform to confirm the matching prior ECG file.
